(19) United States
(12) Patent Application Publication
(10) Pub. No.: US 2018/0370912 A1
(43) Pub. Date: Dec. 27, 2018

(54) COMPOUNDS AND METHODS FOR TREATING DISEASES MEDIATED BY PROTEIN DISULFIDE ISOMERASE

(71) Applicants: Beth Israel Deaconess Medical Center, Inc., Boston, MA (US); The Broad Institute, Inc., Cambridge, MA (US)

(72) Inventors: Robert Flaumenhaft, Newton, MA (US); Partha Pratim Nag, Somerville, MA (US)

(21) Appl. No.: 15/637,597

(22) Filed: Jun. 29, 2017

Related U.S. Application Data
(60) Provisional application No. 62/525,474, filed on Jun. 27, 2017.

(51) Int. Cl. C07D 209/08; A61K 31/4045

(52) U.S. Cl. CPC C07D 209/08; A61K 31/4045

(57) ABSTRACT

Provided herein are compounds of Formula I:

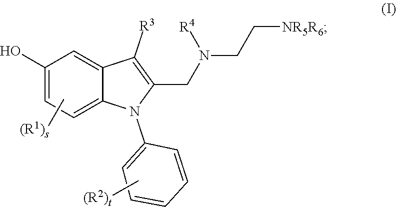

and pharmaceutically acceptable salts and compositions thereof for use in treating diseases associated with the activity or expression of protein disulfide isomerase, wherein the variables are as described herein.

### STATEMENT REGARDING FEDERALLY SPONSORED RESEARCH OR DEVELOPMENT

This invention was supported in part by the United States Government under the National Heart, Lung, and Blood Institute grants HL112302, HL125275, HL112809, and T32 HL007917 and HL116324-02; the National Institute on Drug Abuse grant DA032476; and the National Institutes of Health Molecular Libraries Probe Production Centers Network grant U54 HG005032-1. The Government may have certain rights in this invention.

### BACKGROUND

Protein disulfide isomerase (PDI) is the founding member of a large family of thiol isomerases responsible for catalyzing the folding of nascent proteins in the endoplasmic reticulum. It is a 57 kD oxidoreductase that has an a-b-b′-x-a′ domain structure. (See e.g., Hatahet, F. & Ruddock, L. W. Antioxid Redox Signal 11, 2807-2850 (2009) and Ellgaard, L. & Ruddock, EMBO Rep. 6, 28-32 (2005)). Disulfide shuffling required for protein folding is accomplished by active site cysteines within a CGHC motif in the a and a′ domains. Substrate binding is accomplished by domains b and b′, which contains a deep hydrophobic pocket that supports substrate binding. Although distinct functions have been described for the different domains of PDI, there is cooperativity among them. (See e.g., Freedman, R. B., Klappa, P. & Ruddock, L. W. EMBO Rep. 3, 136-140 (2002) and Darby, N. J., Penka, E. & Vincentelli, R. J Mol Biol 276, 239-247 (1998)). The presence of b and b′ domains in PDI augments reductase activity of a and a′ domains. Conversely, the a and a′ domains are important for binding larger substrates.

In addition to its physiological role in protein folding, PDI has been implicated in a wide variety of pathophysiological processes. PDI expression is upregulated in several cancers (see e.g., Xu, S., Sankar, S. & Neamati, N. Protein disulfide isomerase: a promising target for cancer therapy. Drug Discov. Today 19, 222-240 (2014)) and PDI expression levels correlate with clinical outcomes. (See e.g., McLendon, R. et al. Nature 455, 1061-8 (2008) and Shai, R. et al. Oncogene 22, 4918-23 (2003)). Silencing or inhibition of PDI in animal models of tumor progression suppresses tumor growth and extends survival. (See e.g., Xu, S. et al. Proc Natl Acad Sci USA 109, 16348-16353 (2012) and Yu, S. J. et al. J. Bioenerg. Biomembr. 44, 101-15 (2012)). PDI has also been shown to participate in neurodegenerative processes (see e.g., Uehara, T. et al. S-nitrosylated protein-disulphide isomerase links protein misfolding to neurodegeneration. Nature 441, 513-517 (2006)) and blocking PDI is protective in a cell-based model of Huntington's disease (see e.g., Hoffstrom, B. G. et al. Nat Chem Biol 6, 900-906 (2010) and Kaplan, A. Proc. Natl. Acad. Sci. U.S.A 112, E2245-52 (2015)). Several pathogens subvert extracellular PDI activity to achieve cellular invasion (see e.g., . Khan, M. M. G., Simizu, S., Kawatani, M. & Osada, H, Oncol. Res. 19, 445-53 (2011); Diwaker, D., Mishra, K. P., Ganju, L. & Singh, S. B., Viral Immunol. 28, 153-60 (2015); Walczak, C. P. & Tsai, B., J. Virol. 85, 2386-96 (2011); and Stolf, B. S. et al. ScientificWorldJoumal. 11, 1749-61 (2011). For example, PDI mediates cleavage of disulfide bonds in glycoprotein 120 that are required for HIV-1 entry (see e.g., Fenouillet, E., Barbouche, R., Courageot, J. & Miquelis, R. J. Infect. Dis. 183, 744-52 (2001) and Gallina, A. et al., J Biol Chem 277, 50579-50588 (2002)) and its inhibition interferes with the ability of HIV-1 to infect T cells (see e.g., Bi, S., Hong, P. W., Lee, B. & Baum, L. G., Proc. Natl. Acad. Sci. U.S.A 108, 10650-5 (2011)).

Extracellular PDI also serves a critical role in thrombus formation, the underlying pathology in myocardial infarction, stroke, peripheral artery disease, and deep vein thrombosis. Inhibition of extracellular PDI blocks injury-induced formation of thrombi in multiple animal models of thrombus formation (see e.g., Cho, J., Furie, B. C., Coughlin, S. R. & Furie, B., J Clin Invest 118, 1123-1131 (2008); Jasuja, R. et al., J Clin Invest 122, 2104-2113 (2012); Sharda, A. et al, Blood 125, 1633-4-2 (2015); Furie, B. & Flaumenhaft, R., Circ. Res. 114, 1162-1173 (2014); and Reinhardt, C. et al., J Clin Invest 118, 1110-1122 (2008)). Platelet-specific knockdown of PDI inhibits thrombus formation, demonstrating a role for platelet-derived PDI in thrombus formation (see e.g., Kim, K. et al., Blood 122, 1052-61 (2013)).

Several novel PDI inhibitors have been identified over the past decade. The majority of these antagonists act at the catalytic cysteine within the CxxC motif, blocking all catalytic activity of PDI and most of these antagonists act irreversibly. See e.g., (Flaumenhaft, R., Furie, B. & Zwicker, J. I., Arterioscler. Thromb. Vasc. Biol. 35, 16-23 (2015)). However, because catalytic thioredoxin-like domains of PDI family proteins demonstrate a higher degree of homology than substrate binding domains, which have evolved to associate with different substrate classes, as a class, compounds that interact with the catalytic cysteines of PDI are not selective among thiol isomerases. For example, RL90, a monoclonal antibody that targets PDI, has been shown to cross-react with other closely related thiol isomerases, such as ERp57. See e.g., Wu, Y. et al. Blood 119, 1737-1746 (2012). Similarly, PACMA-31 is also not entirely selective for PDI among thiol isomerases (See FIG. 2).

The substantial homology in the thioredoxin folds of the catalytic domains of thiol isomerases (see e.g., McArthur, A. G. et al, Mol. Biol. Evol. 18, 1455-63 (2001)) continues to complicate efforts to develop compounds that are selective among this large enzyme family. Although, since substrate-binding domains of PDI are less homologous (when compared to the catalytic domains) and the function of PDI relies on cooperative activity of distinct domains, agents which block PDI and do not involve inhibition of the catalytic cysteines, represent an attractive area for the development of selective PDI inhibitors.

### SUMMARY

Provided herein are selective, reversible inhibitors of PDI. Such inhibitors include compounds of Formula I:

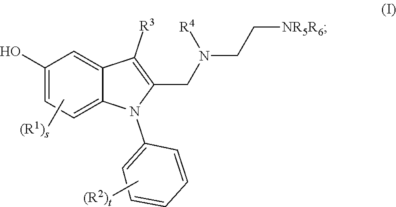

or a pharmaceutically acceptable salt thereof, wherein each of R¹, R², R³, R⁴, R⁵, R⁶, s, and t are as defined herein.

Compounds of Formula I are distinct and have numerous advantages. First, compounds of Formula I were found to target the substrate-binding b′ of PDI and reversibly block substrate binding and inhibit platelet activation and thrombus formation in vivo. (See FIG. 3. Paradoxically, ligation of the substrate-binding pocket by compounds of Formula I enhanced catalytic activity of a and a′. This was surprising and demonstrates a mechanism whereby binding of a substrate to thiol isomerases enhances catalytic activity of remote domains (e.g., structure-function studies showed that displacement of the x-linker by compounds of Formula I act as an allosteric switch to augment reductase activity in the catalytic domains. See FIG. 4). Importantly, although compounds of Formula I target the substrate-binding domain of PDI and elicit augmentation of reductase activity, these compounds demonstrated no such activity when tested against other thiol isomerases (FIG. 7). They also did not inhibit the reductase activity of ERp5, ERp57, or thioredoxin (FIG. 2).

Another advantage of targeting the b′ domain is reversibility. Compounds that target the catalytic domains tend to bind irreversibly via catalytic cysteines. However, by targeting the substrate-binding site, compounds of Formula I act as reversible inhibitors of PDI (FIG. 2; FIG. 9).

As described herein, the provided compounds of Formula I, and pharmaceutically acceptable salts and compositions thereof, are useful for treating a variety of diseases associated with activity or expression of PDI.

### BRIEF DESCRIPTION OF THE FIGURES

FIG. 1 illustrates the effect of bepristat 2a on PDI activity as measured in the insulin turbidimetric assay (dark shades) or the di-eosin-GSSG assay (light shades). Panel (c) illustrates that bepristat 2a (Bep2a) fails to augment activity of a PDI mutant in which the CGHC motif is mutated to AGHA. Panel (d) illustrates the Effect of bepristat 2a on PDI enzyme kinetics. Panel (e) illustrates the effect of quercetin-3-rutinoside (Q-3-R), PACMA-31, or bacitracin on PDI activity as measured by the insulin reductase assay (black) or the di-eosin-GSSG assay (gray).

FIG. 2. illustrates the ability of (a) bepristat 2a (Bep2a) or (b) PACMA-31 and bacitracin to inhibit the reductase activity of PDI (black), ERp5 (red), ERp57 (blue), and thioredoxin (green) as measured in the insulin reductase assay was evaluated. Values represent the percent activity compared with samples exposed to vehicle alone ±SEM (n=2-3). (c) PDI, ERp5, ERp57, ERp72, thioredoxin, or BSA were incubated with vehicle, 300 μM N-ethylmaleimide (NEM), 150 μM quercetin-3-rutinoside (Q-3-R), 150 μM bepristat 2a (Bep2a), 150 μM PACMA-31 or 150 μM bacitracin prior to exposure to MPB. Samples were then analyzed by SDS-PAGE and MPB was detected by Cy5-labeled streptavidin. (d) Washed human platelets (2×10⁸ platelets/ml) were either incubated with vehicle and stimulated with 3 μM SFLLRN (black tracings); incubated with 30 μM bepristat 2a (Bep2a), or 30 μM PACMA-31 for 15 minutes and then stimulated with 3 μM SFLLRN (dark tracings); or incubated with 30 μM bepristat 2a (Bep2a rev), or 30 μM PACMA-31 for 15 minutes, washed, and subsequently stimulated with 3 μM SFLLRN (light tracings). Aggregation was monitored by light transmission aggregometry. Bar graphs represent the average maximal percent aggregation ±SEM (n=3-7). One way ANOVA with Dunnett's post-test: ***p<0.001.

FIG. 3. illustrates (a) platelet-specific anti-GPIN3 antibodies conjugated to Dylight 649 (0.1 μg/g body weight) were infused into mice. Mice were subsequently infused with bepristat 2a (15 mg/kg body weight) as indicated. Thrombi were induced by laser injury of cremaster arterioles before (n=42) and after (n=28) infusion of bepristat 2a. Thrombus formation was visualized by video microscopy for 180 seconds after injury. Representative binarized images of platelets at the injury site before (Control), after bepristat 2a infusion (Bepristat 2a) are shown. Median integrated platelet-fluorescence intensity at the injury site in mice before (dark red) and after (light red) infusion of (c) bepristat 2a infusion is plotted over time.

FIG. 4 illustrates (a) evaluation of domain targets used either full-length PDI or PDI domains including a (residues 1-117), a′ (residues 342-462), ab (residues 1-218), abb′ (residues 1-331), or b′xa′ (residues 219-462) as indicated. Full-length PDI or PDI domains were incubated with vehicle (gray), 30 μM bepristat 2a (blue), or 100 μM PACMA-31 (green) for 30 mins Proteins were then assayed for activity in the insulin reductase assay. Values represent percent vehicle control ±SEM (n=4). (b) Fluorescence monitored at λₑₓ 370 nm following incubation of 50 μM ANS with the isolated a (gold), a′ (green), b (orange), b′x (purple), or vehicle alone (black). (c) PDI or b′x, as indicated, was incubated in the presence of either vehicle (black), 100 μM bepristat 2a (blue), or 100 μM PACMA-31 (green). Samples were then exposed to 50 μM ANS and fluorescence monitored following excitation at λₑₓ 370 nm.

FIG. 5 illustrates (a) full length PDI, b′xa′, and abb′ were incubated with either vehicle (white) or 30 μM bepristat 2a (blue) as indicated. PDI and PDI fragments were subsequently evaluated for activity in the di-eosin-GSSG reductase assay. Values represent percent vehicle control ±SEM (n=3-4). (b) Detection of abb′x by silver staining following incubation with proteinase K for the indicated times in absence and presence of bepristat 2a. (c) Intrinsic fluorescence emission spectra of PDI in the absence (black) or presence of bepristat 2a (blue). (d) SAXS profiles of PDI incubated in the presence of vehicle (black) or bepristat 2b (blue). (e) Plots of the ratio of reduced to oxidized PDI as a function of the ratio of GSH to GSSG in the absence (black) or presence of 50 μM bepristat 2b (blue). The lines represent the best non-linear least squares fit of the data. The calculated equilibrium constants were used to determine the standard redox potentials. Data points are the mean values from analysis of 2-4 peptides encompassing the active site cysteine residues. (f) Fraction of the active site dithiols/disulfides in the reduced state under oxidizing conditions in the absence (black) or presence of 50 μM bepristat 2b (blue). The baseline offsets in the plots shown in part e were a fitted parameter in the non-linear least squares analysis. The error bars represent 1SE.

FIG. 6 illustrates the association of peptides and protein substrates with the substrate-binding domain of PDI and ERp57 augments thiol isomerase activity. PDI (black), ERp5 (red), ERp57 (blue), or ERp72 (green) were incubated in the absence or presence of (a) mastoparan, (b) somatostatin, or (c) cathepsin G at the indicated concentrations and its effect on reductase activity monitored in the di-eosin-GSSG assay. (d) The effect of mastoparan, somatostatin and cathespin G on Kₘ, and k_{cat} was evaluated in the di-eosin-GSSG assay of PDI activity using increasing concentrations of di-eosin-GSSG. (e) Model of substrate-driven allosteric switch that activates thiol isomerase catalytic activity. In the unligated state, the hydrophobic binding pocket within the b′ domain of PDI is capped and disulfide bond formation within the CGHC motif is favored. Binding of substrate to the hydrophobic binding pocket results in displacement of the x-linker and the a′ domain, resulting in a more constrained conformation and favors unpaired cysteines within the CGHC motif. PDI reductase activity is enhanced in the ligated state.

FIG. 7 illustrates the specificity of the bepristat 2a-mediated augmentation of GSSG reductase activity. (a) The effect of 30 μM bepristat 2a on di-eosin-GSSG cleavage was evaluated for full-length PDI and augmentation was quantified. In contrast, bepristats did not augment activity of (b) ERp5, (c) ERp57 or (d) ERp72 in the same assay. Lack of activity of bepristat 2a when used with other thiol isomerases demonstrates the selectively of their augmenting effect on PDI catalytic activity. One way ANOVA with Dunnett's post test: *p<0.05; **p<0.01; ns, non significant.

FIG. 8 illustrates the selectivity of bepristat 2a. (a) PDI, (b) ERp5, (c) ERp57, (d) thioredoxin, or (e) BSA were incubated with vehicle, 300 μM NEM, 150 μM quercetin-3-rutinoside, 150 μM BRD1035, 150 μM BRD4832, 150 μM PACMA-31 or 150 μM bacitracin prior to exposure to MPB. Samples were then analyzed by SDS-PAGE and MPB was detected by Cy5-labeled streptavidin. Fluorescence corresponding to MPB binding was quantified using ImagenQuant 400 analysis software. Values represent mean fluorescence±SEM (n=3-6).

FIG. 9 illustrates the reversibility of bepristats. Reversibility of inhibition of reductase activity of recombinant PDI by Panel (a) bepristat 2a, Panel (b) bepristat 2a or Panel (c) PACMA-31 was evaluated by 100-fold dilution of a mixture of 6 μM bepristat 2a or 300 μM PACMA-31 and monitoring in the insulin turbidimetric assay.

### DETAILED DESCRIPTION

### 1. General Description of the Compounds

In a first embodiment, the present disclosure provides a compound of Formula I:

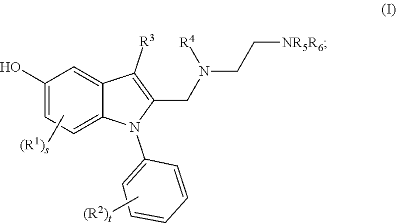

or a pharmaceutically acceptable salt thereof, wherein
R¹ is halo, (C₁-C₄)alkyl, halo(C₁-C₄)alkyl, (C₁-C₄)alkoxy, or halo(C₁-C₄)alkoxy;
R² is halo, (C₁-C₄)alkyl, halo(C₁-C₄)alkyl, (C₁-C₄)alkoxy, or halo(C₁-C₄)alkoxy;
R³ is —C(═O)OR⁷ or C(═O)NR⁸R⁹;
s is 1, 2, or 3;
t is 0, 1, 2, 3, or 4;
R⁴ is hydrogen or (C₁-C₄)alkyl;
R⁵ and R⁶ are each independently hydrogen or (C₁-C₄)alkyl;
R⁷ is (C₁-C₄)alkyl; and
R⁸ and R⁹ are each independently hydrogen or (C₁-C₄)alkyl.

### 2. Definitions

The terms “halo” and “halogen” refers to an atom selected from fluorine (fluoro, —F), chlorine (chloro, —Cl), bromine (bromo, —Br), and iodine (iodo, —I).

The term “alkyl”, used alone or as part of a larger moiety such as e.g., “haloalkyl”, means a saturated monovalent straight or branched hydrocarbon radical having, unless otherwise specified, 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like.

The term “alkoxy”, used alone or as part of a larger moiety such as e.g., “haloalkoxy”, means an alkyl group singular bonded to oxygen thus: —Oalkyl, having, unless otherwise specific, 1-10 carbon atoms and includes, for example, methoxy, ethoxy, propoxy, butoxy, pentyloxy, hexyloxy, heptyloxy, octyloxy, nonyloxy, and decyloxy.

The term “haloalkyl” or “haloalkoxy” includes mono, poly, and perhaloalkyl and perhaloalkoxy groups where the halogens are independently selected from fluorine, chlorine, bromine, and iodine.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts.

The compounds described herein may also be present in the form of a composition, e.g., together with a pharmaceutically acceptable carrier. The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

### 3. Description of Exemplary Compounds

In a second embodiment, the compound of Formula I is of the Formula II:

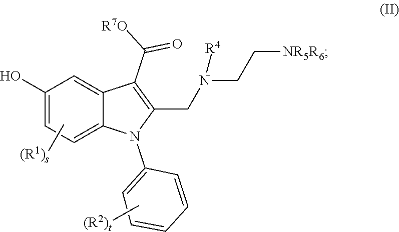

or a pharmaceutically acceptable salt thereof, wherein the variables are as described above for Formula I in the first embodiment.

In a third embodiment, the compound of Formula I is of the Formula III:

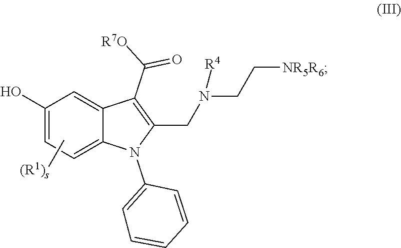

or a pharmaceutically acceptable salt thereof, wherein the variables are as described above for Formula I in the first embodiment.

In a fourth embodiment, the compound of Formula I is of the Formula IV:

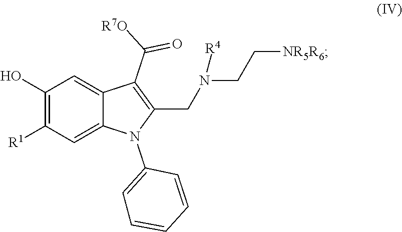

or a pharmaceutically acceptable salt thereof, wherein the variables are as described above for Formula I in the first embodiment.

In a fifth embodiment, the compound of Formula I is of the Formula V:

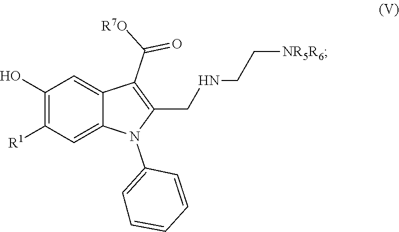

or a pharmaceutically acceptable salt thereof, wherein the variables are as described above for Formula I in the first embodiment.

In a sixth embodiment, R⁵ and R⁶ in Formula I, II, III, IV, or V are both (C₁-C₄)alkyl, wherein the variables are as described above for Formula I in the first embodiment.

In a seventh embodiment, R¹ in Formula I, II, III, IV, or V is halo, wherein the variables are as described above for Formula I in the first embodiment or the sixth embodiment.

In an eighth embodiment, the compound of Formula I is of the formula:

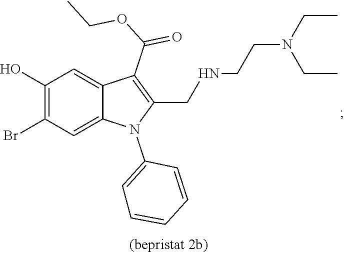 [SMILES: CCOC(=O)c1c(CNCCN(CC)CC)n(-c2ccccc2)c2cc(Br)c(O)cc12]

or a pharmaceutically acceptable salt thereof.

Specific examples of other compounds are provided in the EXEMPLIFICATION. Pharmaceutically acceptable salts as well as the neutral forms of these compounds are included.

### 4. Uses, Formulation and Administration

In one embodiment, provided herein are pharmaceutical compositions comprising a compound described herein; and a pharmaceutically acceptable carrier.

The amount of provided compound that may be combined with carrier materials to produce a composition in a dosage form will vary depending upon the patient to be treated and the particular mode of administration. It will be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided compound in the composition will also depend upon the particular compound in the composition.

In one embodiment, a provided compound or salt thereof, or a provided composition can be used for inhibiting protein disulfide isomerase in a subject in need thereof. This method comprises, e.g., administering to a subject in need thereof, a compound described herein or a pharmaceutically acceptable salt thereof, or a provided composition. In another embodiment, also provided is a compound described herein or a provided composition for use in inhibiting, or for use in the manufacture of a medicament for inhibiting a disease associated with the activity or expression of protein disulfide isomerase.

In another embodiment, a provided compound or salt thereof, or a provided composition can be used for treating a disease associated with the activity or expression of protein disulfide isomerase. This method comprises, e.g., administering to a subject in need thereof, a compound described herein or a pharmaceutically acceptable salt thereof, or a provided composition. In another embodiment, also provided is a compound described herein or a provided composition for use in treating, or for use in the manufacture of a medicament for treating a disease associated with the activity or expression of protein disulfide isomerase.

Diseases that are treatable by the compounds, salts, and compositions described herein include e.g., thrombosis, thrombotic diseases, infectious diseases (e.g., HIV), cancer or inflammation.

In certain embodiments, the thrombotic disease is selected from acute myocardial infarction, stable angina, unstable angina, aortocoronary bypass surgery, acute occlusion following coronary angioplasty or stent placement, transient ischemic attacks, cerebrovascular disease, peripheral vascular disease, placental insufficiency, prosthetic heart valves, atrial fibrillation, anticoagulation of tubing, deep vein thrombosis and pulmonary embolism.

In certain embodiments, the infectious disease is selected from HIV, dengue virus, rotavirus, chlamydia, cytoxicity of diphtheria toxin and phagocytosis of Leishmania chagasi promastigotes. In certain embodiments, the cancer is breast cancer or neuroblastoma.

In certain embodiments, inflammation is selected from inflammation of the lungs, joints, connective tissue, eyes, nose, bowel, kidney, liver, skin, central nervous system, vascular system and heart. Inflammatory lung conditions include, but are not limited to, asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Eye diseases with an inflammatory component include, but are not limited to, uveitis (including iritis), conjunctivitis, scleritis, keratoconjunctivitis sicca, and retinal diseases, including, but not limited to, diabetic retinopathy, retinopathy of prematurity, retinitis pigmentosa, and dry and wet age-related macular degeneration. Inflammatory bowel conditions include Crohn's disease, ulcerative colitis and distal proctitis. Inflammatory skin diseases include, but are not limited to, conditions associated with cell proliferation, such as psoriasis, eczema and dermatitis, (e.g., eczematous dermatitides, topic and seborrheic dermatitis, allergic or irritant contact dermatitis, eczema craquelee, photoallergic dermatitis, phototoxic dermatitis, phytophotodermatitis, radiation dermatitis, and stasis dermatitis).

Other inflammatory skin diseases include, but are not limited to, scleroderma, ulcers and erosions resulting from trauma, burns, bullous disorders, or ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin and cutaneous atrophy resulting from the topical use of corticosteroids. Additional inflammatory skin conditions include inflammation of mucous membranes, such as cheilitis, chapped lips, nasal irritation, mucositis and vulvovaginitis.

Inflammatory disorders of the endocrine system include, but are not limited to, autoimmune thyroiditis (Hashimoto's disease), Type I diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, artherosclerosis, and vascular disease associated with Type II diabetes. Inflammatory condition of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia.

Inflammatory conditions of the liver include, but are not limited to, hepatitis (arising from viral infection, autoimmune responses, drug treatments, toxins, environmental agents, or as a secondary consequence of a primary disorder), biliary atresia, primary biliary cirrhosis and primary sclerosing cholangitis. Inflammatory conditions of the central nervous system include, but are not limited to, multiple sclerosis and neurodegenerative diseases such as Alzheimer's disease, Parkinson's disease, or dementia associated with HIV infection.

Other inflammatory conditions include periodontal disease, tissue necrosis in chronic inflammation, endotoxin shock, smooth muscle proliferation disorders, graft versus host disease, tissue damage following ischemia reperfusion injury, and tissue rejection following transplant surgery. In certain embodiments, the process is blood clotting, platelet aggreagation or fibrin generation.

### EXEMPLIFICATION

The invention now being generally described, it will be more readily understood by reference to the following examples which are included merely for purposes of illustration of certain aspects and embodiments of the present invention, and are not intended to limit the invention.

### General Procedures

### 1. Protein Purification

Recombinant ‘double-tagged’ (Streptavidin-Binding Protein (SBP)-tagged and FLAG-tagged) full-length PDI, ERp57, recombinant His-tagged full-length ERp5, ERp72 and PDI domain fragments were cloned into a pET-15b vector at the NdeI and BamHI sites and transformed into Escherichia coli Origami B (DE3) cells (EMD Chemicals). The recombinant proteins were expressed and isolated by affinity chromatography with complete His-Tag purification resin (Roche Applied Science) or Pierce High Capacity Streptavidin Agarose beads and purified on a Superdex 200 (GE Healthcare).

### 2. Small-Angle X-Ray Scattering (SAXS)

Further purification of full-length PDI was achieved by gel filtration. PDI (1.5, 3.0 and 4.5 mg/mL) was dialyzed against 20 mM Tris, 150 mM NaCl, 5% glycerol (pH: 8.0) containing 0.5 mM bepristat 1b or 2b or DMSO control at 4° C. overnight. Evaluation by SAXS was performed on the SIBYLS beamline in the Advanced Light Source using a high throughput data collection method.

### 3. Redox Potential Determination

The redox potentials of the a (Cys53-Cys56) and a′ (Cys397-Cys400) active-site dithiols/disulfides of human wild-type PDI in the absence or presence of bepristat 2b were determined by differential cysteine alkylation and mass spectrometry. Recombinant PDI (5 μM) was incubated in the absence or presence of bepristat 2b (50 μM) in argon-flushed phosphate-buffered saline containing 0.1 mM EDTA, 0.2 mM oxidized glutathione (GSSG, Sigma) and various concentrations of reduced glutathione (GSH, Sigma) for 18 h at room temperature. Microcentrifuge tubes were flushed with argon prior to sealing to prevent oxidation by ambient air during the incubation period. Unpaired cysteine thiols in PDI and mutants were alkylated with 5 mM 2-iodo-N-phenylacetamide (¹²C-IPA, Cambridge Isotopes) for 1 h at room temperature. The proteins were resolved on SDS-PAGE and stained with SYPRO Ruby (Invitrogen). The PDI bands were excised, destained, dried, incubated with 100 mM dithiothreitol (DTT) and washed. The fully reduced proteins were alkylated with 5 mM 2-iodo-N-phenylacetamide where all 6 carbon atoms of the phenyl ring have a mass of 13 (¹³C-IPA) (Cambridge Isotopes). The gel slices were washed and dried before digestion of proteins with 12 ng/μL of chymotrypsin (Roche) in 25 mM NH₄CO₂ overnight at 25° C. Peptides were eluted from the slices with 5% formic acid, 50% acetonitrile. Liquid chromatography, mass spectrometry and data analysis were performed as described. See e.g., Cook, K. M., McNeil, H. P. & Hogg, P. J., J. Biol. Chem. 288, 34920-9 (2013).

The fraction of reduced active-site disulfide bond was measured from the relative ion abundance of peptides containing ¹²C-IPA and ¹³C-IPA. To calculate ion abundance of peptides, extracted ion chromatograms were generated using the XCalibur Qual Browser software (v2.1.0; Thermo Scientific). The area was calculated using the automated peak detection function built into the software. The ratio of ¹²C-IPA and ¹³C-IPA alkylation represents the fraction of the cysteine in the population that is in the reduced state. The results were expressed as the ratio of reduced to oxidized protein and fitted to equation 1:

$$R = \frac{B + \{ ( 1 - B ) * ( \frac{{[ GSH ]}^{2}}{[ GSSG ]} ) \}}{{K}_{eq} + ( \frac{{[ GSH ]}^{2}}{[ GSSG ]} )} \qquad ( 1 )$$

where R is the fraction of reduced protein at equilibrium, B is the baseline fraction of the cysteine in the population that is in the reduced state and K_{eq} is the equilibrium constant. The standard redox potential (E^{0′}) of the PDI active-site disulfides were calculated using the Nernst equation (equation 2):

$${E}^{0 ′} = {E}_{GSSG}^{0 ′} - \frac{RT}{2 F} ln {K}_{eq} \qquad ( 2 )$$

using a value of −240 mV for the standard redox potential of the GSSG disulfide bond.

### 4. In Vivo Experiment

Intravital video microscopy of the cremaster muscle microcirculation was performed as described previously in Falati, S., Gross, P., Merrill-Skoloff, G., Furie, B. C. & Furie, Nat Med 8, 1175-81. (2002); and Jasuj a, R. et al., J Clin Invest 122, 2104-2113 (2012). Digital images were captured with an Orca Flash 4.0v2 sCMOS camera (Hamamatsu Photonics K.K., Shizuoka Pref., Japan). Representative images are presented, but the median curves include the full data. The kinetics of platelet thrombus formation were analyzed by determining median fluorescence values over time in ˜30-40 thrombi in three mice.

Two hours prior to surgery, 100 mg/kg of the suicide P450 inhibitor 1-aminobenzotriazole (ABT) was administered intraperioneally to each mouse. The cremaster muscle was then surgically exposed. Prior to arteriolar wall injury, DyLight-labeled antibodies were infused intravenously together with drug or vehicle control. Injury to a cremaster arteriolar vessel (30-50-μm diameter) was induced with a MicroPoint laser system Andor Andor Technology, Ltd., Belfast Ireland) focused through the microscope objective, parfocal with the focal plane and tuned to 440 nm through a dye cell containing 5 mM coumarin in methanol. Data were captured digitally from two fluorescence channels, 488/520 nm and 647/670 nm, as well as a brightfield channel Data acquisition was initiated both prior to and following an ablation laser pulse for each injury. The microscope system was controlled and images were collected and analyzed using SlideBook 6.0 (Intelligent Imaging Innovations, Denver, Colo.).

Prior to induction of the thrombus but after injection of the fluorescently labeled antibody, approximately 30 time points were recorded. Subsequently, a thrombus is initiated and recorded for approximately 170 seconds. Post-capture, an upstream region is defined near the site of each thrombus. The maximum pixel intensity in this region is extracted for each time point. The mean of maximal intensity values in the upstream region for each frame is calculated and used as the threshold to define those pixels containing signal. Extracting the values of these pixels and summing them, we obtained the uncorrected integrated intensity for each time point. The area of this region (in pixels) is also reported. Using this information, the actual integrated intensity for each frame is calculated according to the following formula:

ACTUAL INTEGRATED INTENSITY=[sum of the UNCORRECTED INTEGRATED INTENSITY]−[mean of the maxima from the UPSTREAM REGION×area of the UNCORRECTED INTEGRATED INTENSITY]

### 5. Tryptophan Fluorescence

Intrinsic fluorescence spectra were performed in a reaction volume of 50 μL with 5 μM of PDI in 50 mM Tris-HCl buffer containing 150 mM NaCl (pH 7.6). Emission spectra were recorded at 310-400 nm with excitation at 290 nm. Bepristat 2a was at a concentration of 50 μM.

### 6. Maleimide-Polyethanol Glycol-2-Biotin-Binding

Maleimide-Polyethanol glycol-2-Biotin (MPB) binding experiments were performed in a reaction volume of 37.5 μL with 5 μM of thiol isomerase or bovine serum albumin (BSA) in Tris-buffered saline (TBS), in the presence and absence of 1 mM of N-ethylmaleimide (NEM) or bacitracin, and 150 μM of the mentioned other inhibitors. The reaction mixture was incubated at 37° C. for 1 hour. Subsequently, the reaction mixtures were incubated with 25 μM of MPB. The labeling was performed for 20 minutes at 25° C. A total of 12.5 μL of 4× Laemmli Sample Buffer with 5% β-mercaptoethanol was added to each of the samples, followed by heating at 95° C. for 10 minutes. From each sample, 10 μL was loaded on a 12% SDS-PAGE gel, followed by transfer on a nitrocellulose membrane. After blocking the membrane with TBS-T containing 5% BSA for one hour, the membrane was incubated with a 1:2000 dilution of Cy5-tagged Streptavidin for an hour in the dark. Detection of the immunoblots was performed using ImageQuant LAS 4000 and analysis was performed using ImageQuant TL software.

### 7. 1-Anilinonaphthalene-8-Sulfonic Acid fluorescence

The binding of 1-Anilinonaphthalene-8-Sulfonic Acid (ANS) to full length PDI and the different domains of PDI was assessed by incubating 5 μM of protein in the presence or absence of 100 μM of the indicated inhibitors in 175 μL of TBS at 37° C. for 1 hour. Subsequently, 50 μM of ANS was added and the mixture was incubated in the dark at 25° C. for 20 minutes. Fluorescence spectrum (Ex: 370 nm, Em: 400-700 nm) was measured in a 384-well plate. The experiment was performed in triplicate.

### 8. Platelet Aggregation

Platelet aggregation was performed as previously described in Jasuja, R. et al., J Clin Invest 122, 2104-2113 (2012). Briefly, platelet rich plasma (PRP) was obtained from healthy volunteers. Platelets were isolated by centrifugation at 2,000 g and resuspended in Hepes-Tyrode buffer (134 mM sodium phosphate, 2.9 mM KCl, 12 mM sodium bicarbonate, 20 mM HEPES, 1 mM magnesium chloride, 5 mM glucose [pH 7.3]). Washed human platelets (2.5×10⁸ platelets/mL) were incubated with the indicated concentrations of bepristats and PACMA-31 at 37° C. for 20 minutes and then exposed to 3 μM PAR-1 activating peptide SFLLRN. Aggregation was measured using a Chrono-Log 680 Aggregation System.

### 9. Insulin Reductase Assay

The thiol isomerase-catalyzed reduction of insulin was assayed by measuring the increase in turbidity as detected at an optical density (OD) of 650 nm using a Spectramax M3 (Molecular Devices, Sunnyvale, Calif.). The validation assay consisted of 175 nM of PDI in a solution containing 100 mM potassium phosphate (pH 7.4) containing 0.2 mM bovine insulin, 2 mM EDTA, and 0.3 mM DTT (all purchased from Sigma Aldrich, St. Louis, Mo.), inhibitors were used at the concentrations indicated. The reaction was performed at 25° C. for one hour and thirty minutes. For assays of thiol isomerase selectivity, 11 nM PDI, ERp57 or Thioredoxin, or 33 nM ERpS were assayed in similar buffer conditions as described above and inhibitors were used at the indicated concentration. The reaction was performed at 37° C. for forty-five minutes. For assays of isolated domains studies, 400 nM protein was used, except for a, a′ and ab, in which 800 nM protein was used. The assay was performed in similar buffer conditions as described above. Bepristat 2a and N-(2,4-Dimethoxyphenyl)-N-(1-oxo-2-propyn-1-yl)-2-(2-thienyl)glycyl-glycine ethyl ester (PACMA-31) were used at a final concentration of 15 μM in these studies.

Reversibility studies were performed by incubating 20 μM PDI with 6 μM of bepristat 2a or 300 μM of PACMA-31. After 30 minute equilibration; the PDI-inhibitor mixture was diluted 100 fold into the above mentioned assay buffer. The reductase activity of these mixtures were compared to PDI in the presence or absence of 6 μM bepristat 2a or 0.06 μM bepristat 2a or 300 μM PACMA-31 or 3 μM PACMA-31.

### 10. Di-Eosin-GSSG Disulfide Reductase Assay

The probe di-eosin glutathione disulfide, di-eosin-GSSG, was prepared as previously described in Raturi, A., Vacratsis, P. O., Seslija, D., Lee, L. & Mutus, B., Biochem J 391, 351-357 (2005). Reductase activity of purified thiol isomerases and PDI domains was monitored in a 96-well fluorescence plate format. PDI, AGHA-PDI, ERpS, ERp57, ERp72 and PDI domains were assayed at 50 nM in the presence or absence of the indicated small molecules, peptides or cathepsin G. The assay included 100 mM potassium phosphate (pH 7.4) containing 2 mM EDTA, 5 μM DTT and 150 nM of the di-eosin GSSG probe. The increase in fluorescence was determined for 20 minutes by excitation at 520 nm and emission at 550 nm in a Synergy Biotek 4. The reduction of 150 nM di-eosin-GSSG by 5 μM DTT in the presence or absence of mentioned small molecules, peptides or proteins served as a negative control.

Michaelis Menten analysis was assayed using PDI at 20 nM and small molecules, peptides or cathepsin G at concentrations causing maximal augmentation under similar buffer conditions as described above. 8-point response curves were generated using a range of different di-eosin-GSSG concentrations (50-5000 nM). Enzyme kinetics analysis was performed using Graphpad Prism 5.0.

### 11. Proteolysis Assay

Proteinase K (2 μg/mL) was incubated with 1.25 μg of the abb′x fragment in 50 mM Tris-HCl containing 5 mM CaCl₂ and 10 mM DTT. Reactions were aborted with the addition of 0.5 mM phenylmethanesulfonyl fluoride (PMSF). Subsequently, samples were subjected to Laemmli Sample buffer with 5% β-mercaptoethanol and heated at 95° C. for 10 minutes. Each sample was loaded on a 12% SDS-PAGE gel, followed by silver staining using the Pierce Silver Stain Kit (Thermo Scientific).

### Results/Discussion

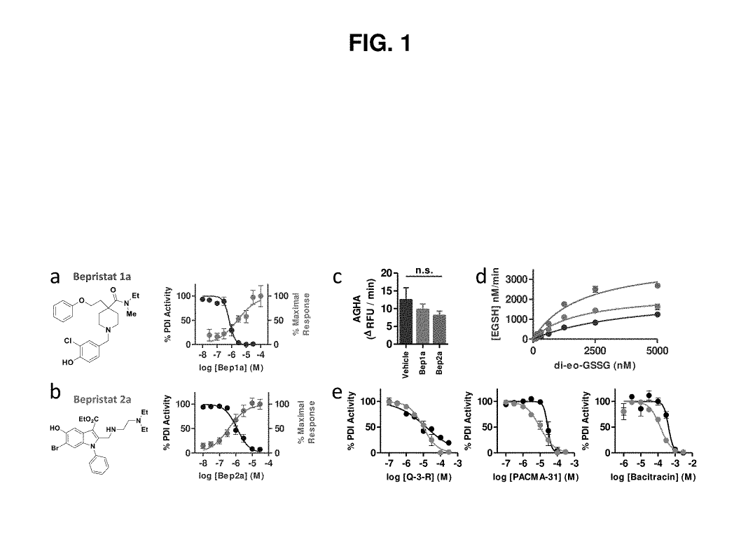

Bepristat 2a was found to have an IC₅₀ of ˜1.2 μM (FIG. 1b). Interestingly, despite potent PDI inhibitory activity in the insulin reductase assay, bepristat 2a did not inhibit in a di-eosin-GSSG-based assay that measures reductase activity at the catalytic cysteines as in Raturi, A. & Mutus, B., Free Radic Biol Med 43, 62-70 (2007) (See FIGS. 1a and 1b). Rather than inhibiting reductase activity at the catalytic cysteines, bepristat 2a enhanced cleavage of the di-eosin-GSSG probe by PDI (FIGS. 1a and 1b). Bepristat 2a did not affect the fluorescence of di-eosin-GSSG in the absence of PDI, nor did bepristat 2a affect di-eosin-GSSG fluorescence in the presence of a catalytically inactive PDI (FIG. 1c). Michaelis-Menten analysis performed in the absence of bepristats showed an apparent K_{M} of 4168 nM and an apparent k_{cat} of 1135 min⁻¹. Incubation with bepristat 2a decreased the apparent K_{M} to 1949 nM and increased the apparent k_{cat} to 1995 min⁻¹ (FIG. 1d). Bepristat 2a demonstrated more potent inhibitory activity in the insulin turbidimetric assay when compared with quercetin-3-rutinoside (rutin), a glycosylated flavonoid quercetin shown to block PDI activity and inhibit thrombus formation in vivo (see Jasuja, R. et al., J Clin Invest 122, 2104-2113 (2012) and Lin, L. et al., J. Biol. Chem. (2015)); PACMA-31, a irreversible inhibitor of PDI that binds the catalytic cysteine and impairs tumor growth in a murine model ovarian cancer (see Xu, S. et al., Proc Natl Acad Sci USA 109, 16348-16353 (2012)); and bacitracin, a dodecapeptide that has been used for decades as the standard PDI inhibitor (see Mandel, R., Ryser, H. J., Ghani, F., Wu, M. & Peak, D., Proc Natl Acad Sci USA 90, 4112-4116 (1993)) (FIG. 1e). Yet these other inhibitors all blocked PDI-mediated cleavage of di-eosin-GSSG when used at concentrations required to inhibit activity in the insulin turbidimetric assay (FIG. 1e). These results indicate that bepristat 2A blockS PDI activity via a mechanism that differs from that of previously described PDI inhibitors. Values for the insulin turbidimetric assay represent percent of PDI activity compared with a control exposed to vehicle alone, mean±SEM (n=3−6). Values for the di-eosin assay represent the rate of di-eosin-GSSG fluorescence generation measured for 20 min ±SEM (n=3).

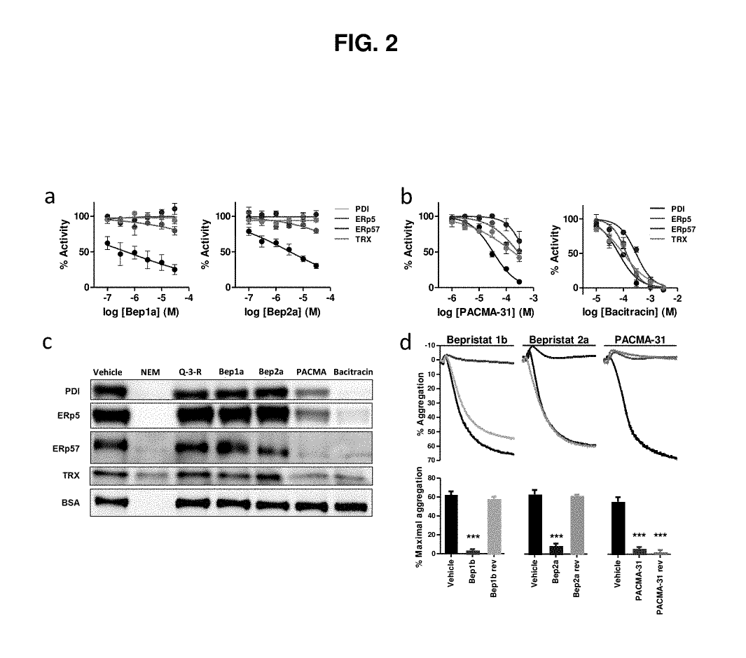

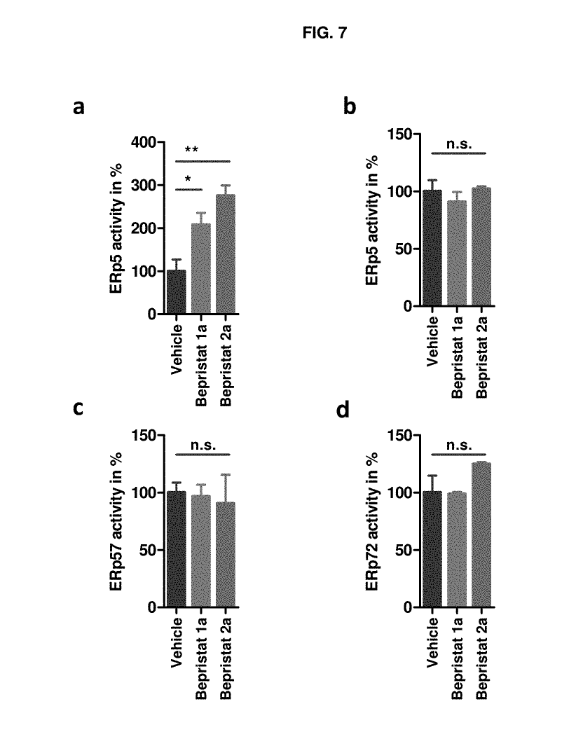

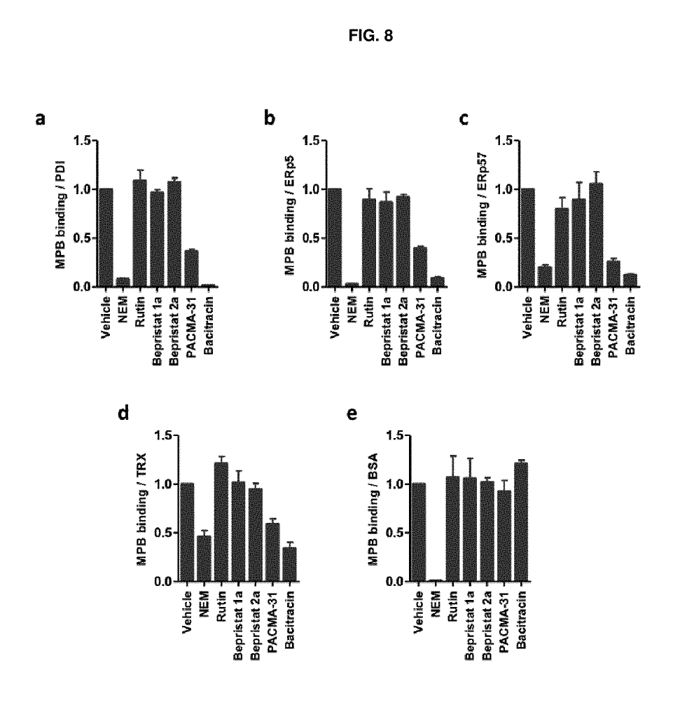

Bepristat 2a was selective among vascular thiol isomerases, even at concentrations 10-fold higher than its IC₅₀ (FIG. 2a; FIG. 7). In contrast, PACMA-31 demonstrated inhibitory activity against both ERp5 and T×R when used at 10-fold their IC₅₀ (FIG. 2b). Bacitracin showed activity against all thiol isomerases tested. Both PACMA-31 and bacitracin interfered with the reaction of maleimide-polyethylene glycol-2-biotin (MPB) with the catalytic cysteines of ERp5, ERp57, and thioredoxin (FIG. 2c; FIG. 8) demonstrating lack of selectivity by virtue of non-selective interactions with catalytic cysteines on thiol isomerases. None of the PDI inhibitors interfered with the MPB reaction with BSA, demonstrating that the inhibitors are not non-selectively reacting with MPB or with any free cysteine.

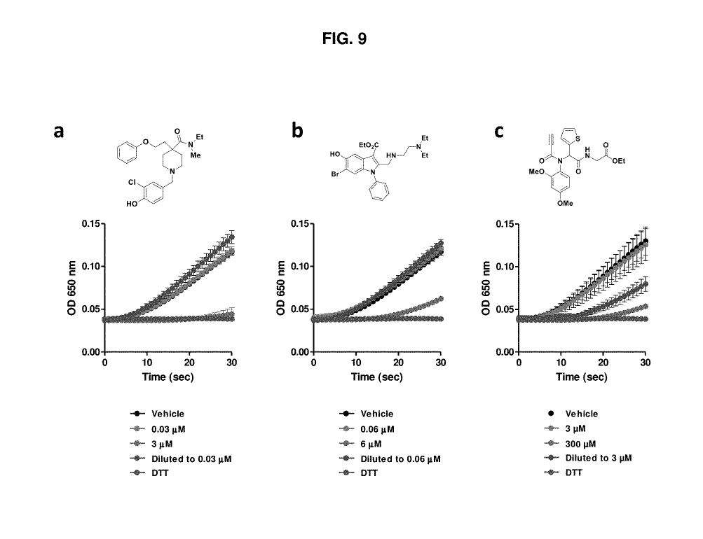

Inhibition of PDI by either blocking antibodies or small molecule inhibitors interferes with agonist-induced platelet activation (see e.g., Cho, J., Furie, B. C., Coughlin, S. R. & Furie, B. A critical role for extracellular protein disulfide isomerase during thrombus formation in mice. J Clin Invest 118, 1123-1131 (2008) and Jasuja, R. et al. Protein disulfide isomerase inhibitors constitute a new class of antithrombotic agents. J Clin Invest 122, 2104-2113 (2012)). Platelet-specific knockdown of PDI also impairs platelet activation (see e.g., Kim, K. et al. Platelet protein disulfide isomerase is required for thrombus formation but not for hemostasis in mice. Blood 122, 1052-61 (2013)). We determined whether blocking PDI using bepristat 2a inhibits platelet activation. Platelets were incubated with either bepristat bepristat 2a or PACMA-31 and their response to the PAR1 peptide agonist, SFLLRN, evaluated by light transmission aggregometry. Bepristat 2a and PACMA-31 all inhibited platelet aggregation (FIG. 2d). To evaluate reversibility of inhibition using the platelet aggregation assay, platelets were incubated with PDI antagonists for 30 minutes, washed, and then stimulated with SFLLRN. Inhibition of platelet aggregation by bepristat 2a was restored following washing. In contrast, platelet aggregation by PACMA-31 was irreversibly inhibited under these conditions (FIG. 2). To confirm that bepristat 2a is a reversible inhibitors of PDI, we evaluated reversibility in the insulin reductase assay. These studies demonstrated that the inhibitory effect of bepristat 2a was readily reversed by dilution to a subinhibitory concentration, while that of PACMA-31 was not (FIG. 9).

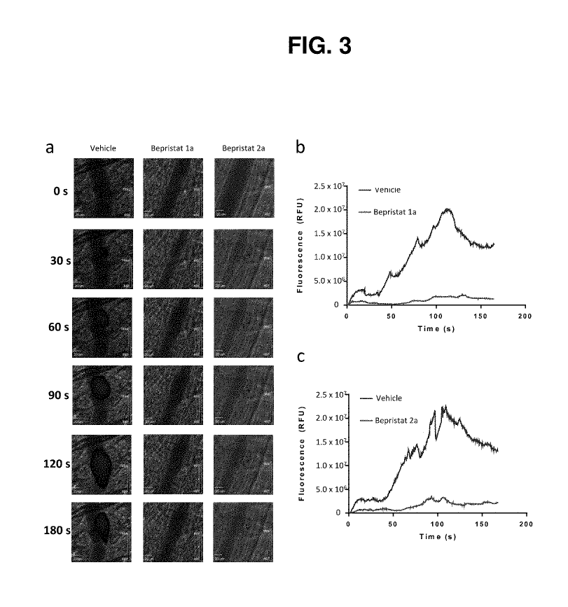

Inhibition of PDI using anti-PDI antibodies or by small molecules such as bacitracin or quercetin-3-rutinoside inhibits thrombus formation in vivo. Similarly, platelet-selective deletion of PDI interferes with thrombus formation in mouse arterioles following vascular injury. We therefore evaluated the effect of bepristats on thrombus formation in cremaster arterioles following laser-induced vascular injury (FIG. 3a). Bepristat 2a infusion inhibited platelet accumulation at sites of laser-induced injury by 14.9% of controls (p=0.02; FIG. 3c). These results demonstrate that bepristat 2a is tolerated in vivo and potently inhibits thrombus formation.

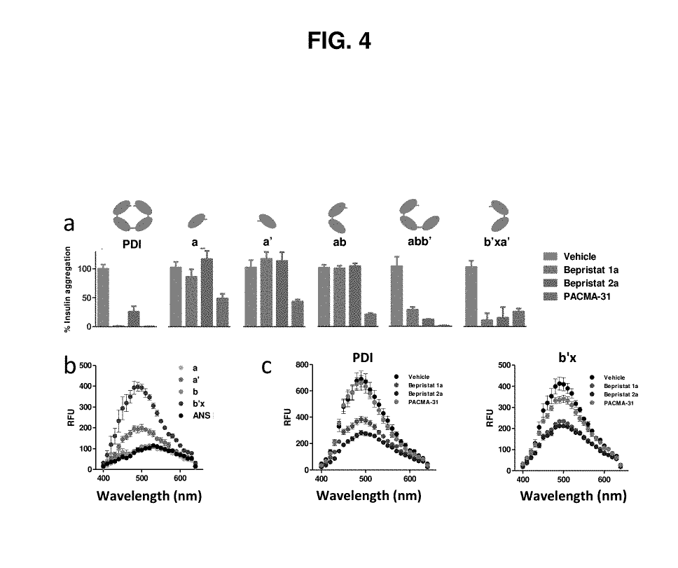

To determine the mechanism by which bepristat 2a modulates PDI activity, we tested the compounds against PDI fragments containing the a or a′ domains using the insulin turbidimetric assay. These fragments included the a domain, a′ domain, ab domains, abb′ domains, and b′xa′ domains (FIG. 4a). Although the isolated domains had diminished insulin reductase activity compared to full-length PDI, their activity could be quantified and the effects of antagonists on their activity tested. Bepristat 2a did not have activity against the isolated a domain, a′ domain, or ab domain (FIG. 4a). In contrast, Bepristat 2a blocked activity of the abb′ and b′xa′ domains. PACMA-31 inhibited reductase activity of all PDI fragments in the insulin turbidimetric assay (FIG. 4a). These results demonstrate that bepristat 2a inhibitS PDI reductase activity by binding outside the catalytic motif, at b′.

The C-terminal end of the b′ domain is connected to an x-linker that covers a deep hydrophobic pocket in b′ and is thought to mediate the movement of the a′ domain relative to the rest of the protein. The x-linker consists of a flexible 19 amino acid peptide that can adapt at least two conformations. One is a ‘capped’ conformation in which the x-linker covers the hydrophobic pocket. PDI can also assume an ‘uncapped’ conformation in which the x-linker is displaced from the hydrophobic binding site. In a b′x fragment in which isoleucine 272 is mutated to alanine, the x-linker is constitutively associated with the hydrophobic patch on the b′ domain. 8-anilinonaphthalene-1-sulfonic acid (ANS) fluorescence was used to evaluate binding to hydrophobic regions on PDI in wild type and mutant constructs. Binding of ANS to hydrophobic regions results in a marked increase in fluorescence when evaluated at λₑₓ 370 nm (FIG. 4b). ANS fluorescence was prominent upon incubation with the isolated b′x domain and weak upon incubation with isolated a, a′, or b domains (FIG. 4b). ANS fluorescence was not observed upon incubation with I272A mutant, indicating that obstruction of binding pocket by the x-linker prevented ANS binding. Bepristat 2a interfered with the increase in ANS fluorescence observed upon incubation with PDI (FIG. 4c). In contrast, incubation with PACMA-31 failed to block ANS fluorescence (FIG. 4c). Similar results were obtained when binding of ANS to the isolated b′x domain was evaluated (FIG. 4c).

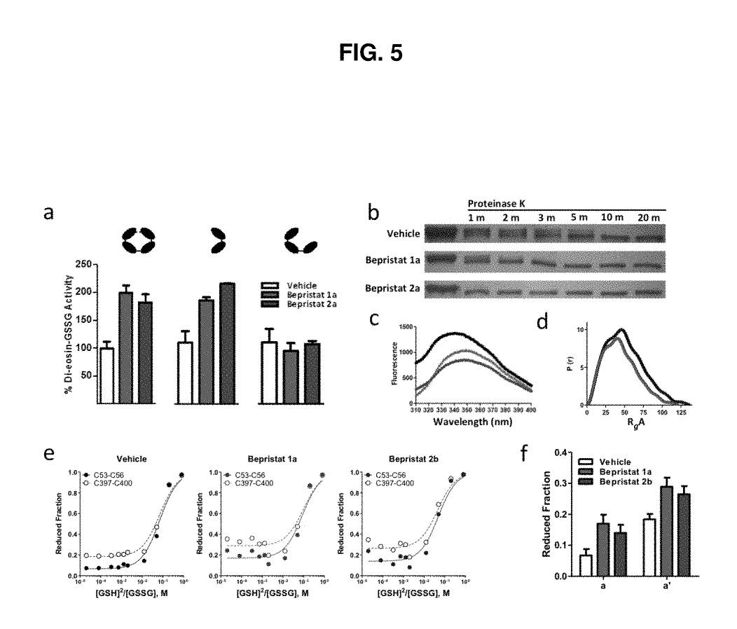

Bepristat 2a binds the b′ domain and enhance catalytic activity at the a and a′ domains, raising the question of how binding of a small molecule to one domain modifies enzymatic activity at remote domains. In order to determine which domains contribute to the ability of bepristat 2a to augment activity at the catalytic cysteines of PDI, the effect of bepristat 2a on di-eosin-GSSG cleavage was tested using full-length PDI, abb′, and b′xa′. Bepristat 2a significantly augmented the catalytic activity of the b′xa′ fragment (FIG. 5a). In contrast, bepristat 2a failed to augment activity of the abb′ fragment, which is missing the x-linker.

The observation that bepristat 2a targets the same hydrophobic pocket on the b′ domain that the x-linker associates with suggested that binding of bepristats results in displacment of the x-linker. To evaluate this possibility, we tested the protease sensitivity of abb′x in the presence and absence of bepristats. Proteinase K cleaves PDI from the C-terminal end. Cleavage of abb′x by proteinase K occurred more rapidly in the presence of bepristat 2a than in its absence (FIG. 5b). bepristat 2a did not interfere with proteinase K activity as evidenced by the fact that proteinase K cleaved ERp5 equally well in the presence or absence of bepristat 2a (not shown). The effect of bepristat 2a on movement of the x-linker was also evaluated using intrinsic fluorescence measurements. The x-linker includes a tryptophan residue (Trp-347) that associates with the hydrophobic binding pocket on the b′ domain, resulting in an increase in intensity and a blue shift. Incubation with bepristats resulted in a loss of intensity of intrinsic tryptophan fluorescence and a red shift (FIG. 5c), indicating that bepristats elicit movement of the x-linker upon incubation with b′x. These results support a model whereby ligation of bepristats at the hydrophobic binding pocket results in displacement of the x-linker.

Small angle x-ray scattering (SAXS) was used to determine whether the local effects of bepristat 2a on interactions between the b′ domain and the x-linker had global consequences on PDI conformation. PDI is a flexible protein that exhibits dynamic behavior in aqueous solution. We used SAXS to measure the gyration radius (Rg) of PDI in aqueous solution. An overall molecular envelope of PDI was derived from these measurements. We found the gyration radius for full-length oxidized PDI to be 40.0 Å, whereas the gyration radius of 35.3 Å and PDI complexed with bepristat 2b showed a gyration radius of 35.9 Å (FIG. 5d). These data suggest that bepristat 2a constrainS the dynamic behavior of PDI. In the presence of bepristat 2a, PDI adopts a more compact conformation that closely approximates the overall envelope of reduced PDI.

By binding the b′ domain and eliciting a change in the global conformation in PDI, bepristat 2a could modify disulfide bond formation at the CGHC motifs. Differential cysteine alkylation followed by mass spectroscopy under varying GSH:GSSG ratios was used to quantify unpaired thiols and disulfide bonds within the CGHC motifs of the a and a′ domains. This technique uses ¹²C-IPA to label unpaired thiols. Disulfide bonds are then reduced using DTT and the resulting free thiols are labeled using ¹³C-IPA. Differential cysteine alkylation showed that in a relatively oxidizing environment (high GSSG to GSH ratio), the fraction of reduced PDI was increased following incubation with bepristats, as indicated by the baseline offset (FIG. 5 e,f). This difference between samples exposed to bepristats and control samples is lost under reducing conditions (high GSH to GSSG ratio). Calculation of the redox potential of the two active sites demonstrated no difference between bepristat-exposed and control samples, since the effect of bepristat 2a is overcome under more reducing conditions. However, these data show that the conformational change induced by bepristat 2a impairs active site disulfide bond formation under equilibrium conditions.

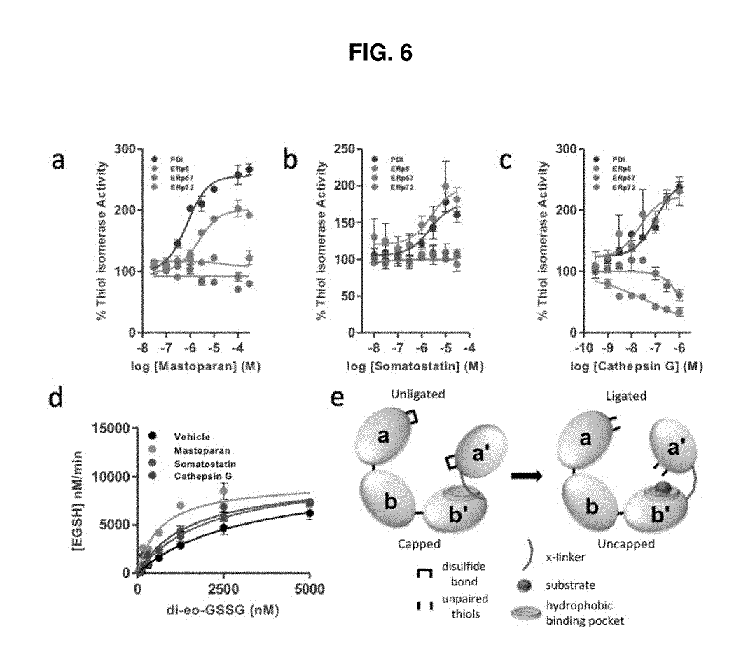

Bepristat 2a affects PDI activity by a previously unrecognized mechanism involving engagement of a hydrophobic pocket on the b′ domain with displacement of the x-linker resulting in a conformation change and enhanced reductase activity. Peptides such as mastoparan have previously been shown to associate with the hydrophobic pocket on b′. Incubation of PDI with mastoparan enhanced the ability of PDI to cleave di-eosin-GSSG in a dose-dependent manner (FIG. 6a), even though mastoparan inhibits PDI activity in an RNase refolding assay. Somatostatin, a 14 amino-acid peptide hormone, also binds PDI and stimulates cleavage of di-eosin-GSSG (FIG. 6b). In addition, a protein substrate of PDI, cathepsin G, elicits enhanced reductase activity (FIG. 6c). Like bepristat 2a, mastoparan, somatostatin, and cathepsin G all decreased the apparent K_{M} in the di-eosin-GSSG assay of PDI reductase activity (FIG. 6d). None of them altered the apparent k_{cat}.

Peptides and protein substrates associate with substrate binding domains of other thiol isomerases. To evaluate whether substrate-driven augmentation of catalytic activity is observed in other thiol isomerases, we tested the ability of mastoparan, somatostatin, and cathepsin G to enhance cleavage of di-eosin-GSSG by ERp72, ERp57, and ERp5. All three substrates augmented the catalytic activity of ERp72 (FIG. 6a-c). In contrast, neither mastoparan, somatostatin, nor cathepsin G was able to augment the catalytic activity of ERp57 or ERp5. In fact, these substrates inhibited the catalytic activity of ERp5 and ERp57 to varying degrees (FIG. 6a-c).

All publications and patents cited herein are hereby incorporated by reference in their entirety.

1. A compound of Formula I: 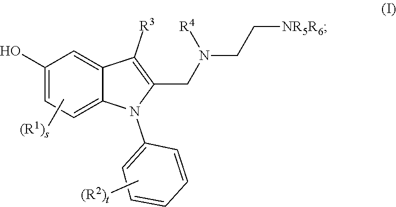
or a pharmaceutically acceptable salt thereof, wherein
R¹ is halo, (C₁-C₄)alkyl, halo(C₁-C₄)alkyl, (C₁-C₄)alkoxy, or halo(C₁-C₄)alkoxy;
R² is halo, (C₁-C₄)alkyl, halo(C₁-C₄)alkyl, (C₁-C₄)alkoxy, or halo(C₁-C₄)alkoxy;
R³ is —C(═O)OR⁷ or C(═O)NR⁸R⁹;
s is 1, 2, or 3;
t is 0, 1, 2, 3, or 4;
R⁴ is hydrogen or (C₁-C₄)alkyl;
R⁵ and R⁶ are each independently hydrogen or (C₁-C₄)alkyl;
R⁷ is (C₁-C₄)alkyl; and
R⁸ and R⁹ are each independently hydrogen or (C₁-C₄)alkyl.

2. The compound of claim 1, wherein the compound is of the Formula II: 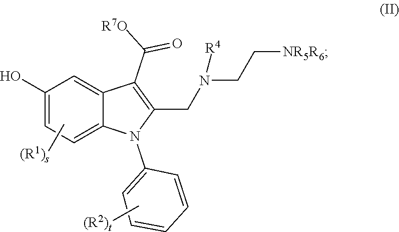
or a pharmaceutically acceptable salt thereof.

3. The compound of claim 1, wherein the compound is of the Formula III: 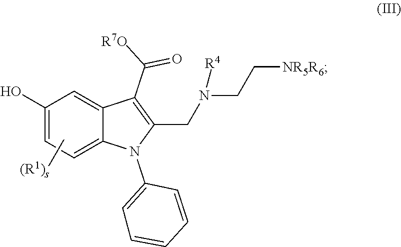
or a pharmaceutically acceptable salt thereof.

4. The compound of claim 1, wherein the compound is of the Formula IV: 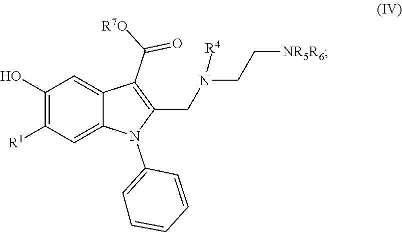
or a pharmaceutically acceptable salt thereof.

5. The compound of claim 1, wherein the compound is of the Formula V: 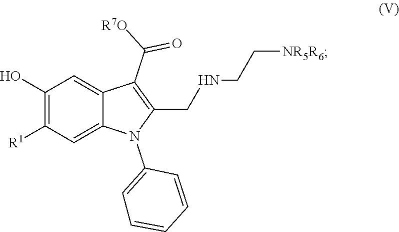
or a pharmaceutically acceptable salt thereof.

6. The compound of claim 1, wherein R⁵ and R⁶ are both (C₁-C₄)alkyl.

7. The compound of claim 1, wherein R¹ is halo.

8. The compound of claim 1, wherein the compound is of the formula: 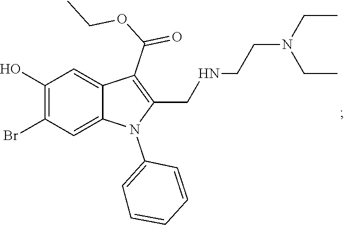 [SMILES: CCOC(=O)c1c(CNCCN(CC)CC)n(-c2ccccc2)c2cc(Br)c(O)cc12]
or a pharmaceutically acceptable salt thereof.

9. A pharmaceutical composition comprising a compound according to claim 1; and a pharmaceutically acceptable carrier.

10. A method of inhibiting protein disulfide isomerase in a subject in need thereof, comprising administering to the subject a compound of claim 1.

11. A method of treating a disease associated with the activity or expression of protein disulfide isomerase, comprising administering to a subject in need thereof a compound of claim 1.
